(19) United States
(12) Patent Application Publication
(10) Pub. No.: US 2015/0298111 A1
(43) Pub. Date: Oct. 22, 2015

(54) Ruthenium Or Osmium Complex, Method For Its Preparation And Use Thereof

(71) Applicants: Tomasz WDOWIK, Rzeszow (PL); Cezary SAMOJLOWICZ, Warszawa (PL); Magdalena JAWICZUK, Miedzyrzecz Podlaski (PL); Karol GRELA, Warszawa (PL)

(72) Inventors: Tomasz WDOWIK, Rzeszow (PL); Cezary SAMOJLOWICZ, Warszawa (PL); Magdalena JAWICZUK, Miedzyrzecz Podlaski (PL); Karol GRELA, Warszawa (PL)

(73) Assignee: Instytut Chemii Organicznej Polskiej Akademii Nauk, Warzsawa (PL)

(21) Appl. No.: 14/346,865

(22) PCT Filed: Sep. 23, 2012
(86) PCT No.: PCT/IB2012/055058

§ 371 Date: Mar. 24, 2014

(30) Foreign Application Priority Data
Sep. 26, 2011 (PL) P.396439

(51) Int. Cl. B01J 31/22; C07C 67/333; C08G 61/08; C07C 29/56; C07C 67/297; C07D 307/28; C07F 15/00; C07D 207/48

(52) U.S. Cl. CPC B01J 31/2269; C07F 15/0046; C07C 67/333; C07D 207/48; C07C 29/56; C07C 67/297; C07D 307/28; C08G 61/08; B01J 2231/52; B01J 2231/54; B01J 2531/821; C08G 2261/3324; C08G 2261/418

(57) ABSTRACT

The subject of the present invention are novel metal complexes defined by Formula 1:

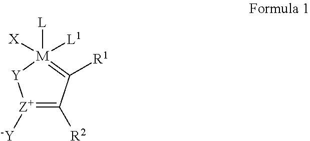

The present invention also relates to methods of producing said novel metal complexes defined by Formula 1 as well as their uses.

This application is a National Stage Application of PCT/IB2012/055058, filed Sep. 23, 2012.

The present invention relates to novel complexes of metals that act as pre(catalysts), a method of preparation them as well as their use in the metathesis, isomerisation and cycloisomerisation of olefins, and cycloisomerisation reactions, as well as in olefin as well as in hydrogen transfer. The present invention is useful in broadly understood organic synthesis.

The use of olefin metathesis in organic synthesis has recently seen much progress. The state of the art reveals several carbene complexes of ruthenium acting as (pre)catalysts which possess both high activity in metathesis reactions of various kinds, as well as a broad tolerance of functional groups. The above combination of properties warrants the utility of these types of (pre)catalysts in organic synthesis.

From the point of view of practical use, particularly on an industrial scale, it is very desirable that such ruthenium complexes are stable, for extended periods at elevated temperatures, and may be stored and/or purified and/or used without an inert gas atmosphere. It is also important that these catalysts exhibit tunable reactivity, depending on the reaction conditions, and that they are easy to remove after the reaction.

Many complexes of ruthenium active in olefin metathesis have been disclosed (see: Org. Lett. 1999, 1, 953-956; J. Chem. Soc. Chem. Commun. 1999, 601-602). It is also known that increased stability is connected with decreased catalytic activity (for comparison: J. Am. Chem. Soc. 2000, 122, 8168-8179; Tetrahedron Lett. 2000, 41, 9973-9976). These types of advantages and limitations have also been noted in the case of (pre)catalysts activated by steric or electron factors of the benzylidene ligands (for a comparison of catalytic activity see: Angew. Chem. Int. Ed. 2002, 114, 4210-4212; Angew. Chem. Int. Ed. 2002, 114, 2403-2405).

The effect of anionic ligands has also been demonstrated (see: Angew. Chem. Int. Ed. 2007, 46, 7206-7209; Organometallics, 2010, 29, 6045-6050; Organometallics, 2011, 30, 3971-3980) as well as of NHC ligands (see: Chem. Rev. 2010, 110, 1746-1787; Chem. Rev. 2009, 109, 3708-3742) on the activity and selectivity of (pre)catalysts. From these reports, it is known that the exchange of a chloride ligand for an oxyacid residue increases the stability of the (pre)catalyst, at the same time decreases catalytically activity.

Unexpectedly it was shown that the novel ruthenium complexes according to the present invention defined by Formula 1:

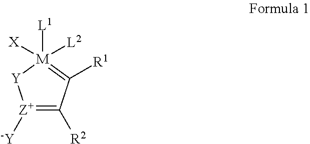

which contains a chelate ring formed by an oxygen atom are thermally stable and exhibit good catalytic activity. Additionally, these compounds significantly alter the selectivity of the reaction depending on the use of: a solvent and/or the addition of an acid or halide derivatives of alkanes or halide derivatives of silanes or N-haloimides or N-haloamides; which enables the control over the catalytic processes through the exchange of these factors.

Complexes defined by Formula 1, according to the present invention are useful in a broad range of reactions. A good result may be obtained by conducting both numerous metathesis ring closure reactions, as well as homometathesis, cross-metathesis as well as metathesis of the “alkene-alkyne” (ene-yne), ring-opening polymerisation reactions (ROMP), olefin isomerisation reactions, olefin cycloisomerisation reactions as well as hydrogen transfer reactions.

The high polarity of the compounds being the subject of the present invention also makes it easier to remove ruthenium compounds from the reaction products, which is very significant in the synthesis of compounds for the pharmaceutical industry.

The subject of the present invention are novel metal complexes, containing a nitroanion group defined by Formula 1:

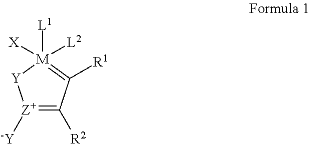

in which:
M denotes ruthenium or osmium;
L¹ and L² denote neutral ligands;
X denotes an anionic ligand;
Z denotes a nitrogen atom;
Y denotes an oxygen atom;
R¹, R² denote, independently of one another, a hydrogen atom, a fluoride atom, C₁-C₂₅ alkyl, C₁-C₂₅ perfluoroalkyl, C₂-C₂₅ alkene, C₃-C₇ cycloalkyl, C₂-C₂₅ alkenyl, C₃-C₂₅ cycloalkenyl, C₂-C₂₅ alkynyl, C₃-C₂₅ cycloalkynyl, C₁-C₂₅ alkoxyl, C₅-C₂₄ aryl, C₅-C₂₀ heteroaryl, or a 3-12 membered heterocycle wherein the alkyl groups may be joined together in a ring, preferentially a hydrogen, a nitro (—NO₂), cyanide (—CN), carboxyl (—COOH), ester (—COOR′), amido (—CONR′₂), sulphonyl (—SO₂R′), formyl (—CHO), sulphonoamido (—SO₂NR′₂), or ketone (—COR′) group, in which R has the following meaning: C₁-C₅ alkyl, C₁-C₅ perfluoroalkyl, C₅-C₂₄ aryl.

In a preferable embodiment R¹ of Formula 1 denotes a hydrogen atom or methyl group; R² denotes a hydrogen atom and the

anionic ligand X denotes a fluoride atom, a —CN, —SCN, —OR⁴, —SR⁴, —O(C═O)R⁴, —O(SO₂)R⁴, or —OSiR₃₄ group, where R⁴ denotes an C₁-C₁₂ alkyl, C₃-C₁₂ cycloalkyl, C₂-C₁₂ alkenyl, or C₅-C₂₀ aryl, which may be substituted with at least one C₁-C₁₂ alkyl, C₁-C₁₂ perfluoroalkyl, C₁-C₁₂ alkoxyl or fluoride atom; and
the neutral ligands L¹ and L² are selected, independently of one another, from a group encompassing —P(R⁵)₃, —P(OR⁵)₃ or N-heterocyclic carbene ligands denoted by Formulae 2a, 2b, 2c, 2d, 2e, 2f, 2g, 2h, 2i, 2j, 2k, 2l, 2m, 2n, 2o or 2p:

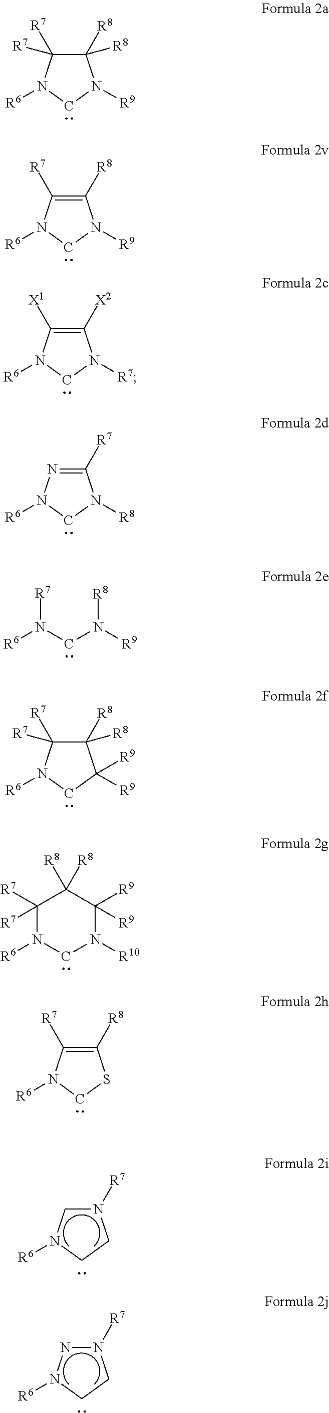

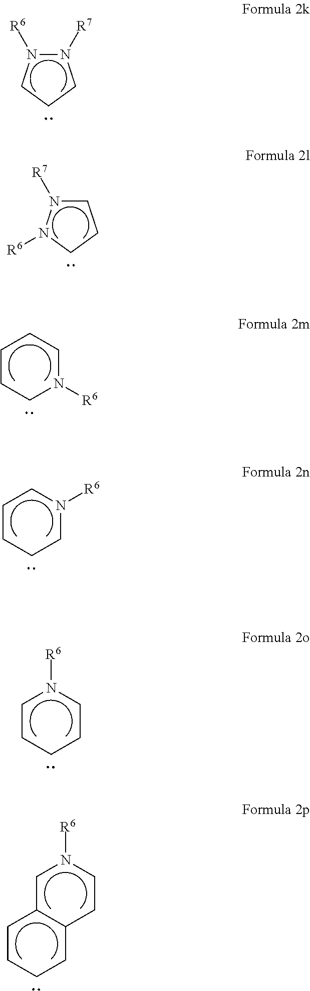

where:
each R⁵ denotes, independently of one another, C₁-C₁₂ alkyl, C₃-C₁₂ cycloalkyl, C₅-C₂₀ aryl, 5-12 membered heteroaryl;
each R⁶, R⁷, R⁸, R⁹ and R¹⁰ denotes, independently of one another, a hydrogen atom, C₁-C₁₂ alkyl, C₃-C₁₂ cycloalkyl, C₂-C₁₂ alkenyl or C₅-C₂₀ aryl which may be substituted with at least one C₁-C₁₂ alkyl, C₁-C₁₂ perfluoroalkyl, C₁-C₁₂ alkoxyl or fluoride atom, and groups R⁶, R⁷, R⁸, R⁹ and R¹⁰ may possibly be interconnected.

Carbene ligands may be classically coordinated, as in structures 2a-2h, or in a non-classic fashion (“abnormal carbenes,—see: Chem. Rev. 2009, 109, 3445) as in structures 2i-2p.

In another preferable embodiment, the anionic ligand X of Formula 1 denotes a chlorine atom; and
neutral ligand L¹ denotes —P(R⁵)₃ in which substituent R⁵ has a meaning as set out above; and
neutral ligand L² denotes ligands defined by Formula 2a or 2b:

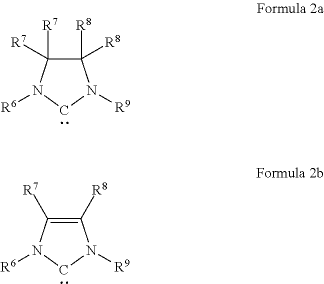

in which substituents R⁶, R⁷, R⁸ and R⁹ mean as defined above.

The subject of the present invention is also a method of producing complexes of metals defined by Formula 1, which encompasses the reaction of compounds defined by Formula 3

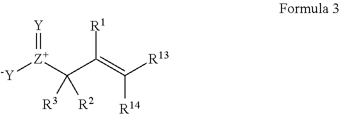

in which R¹, R², Z, Y have meanings as defined above, whereas R³, R¹³, R¹⁴ denote, independently of one another, a hydrogen atom, a fluoride atom, C₁-C₂₅ alkyl, C₁-C₂₅ perfluoroalkyl, C₂-C₂₅ alkene, C₃-C₇ cycloalkyl, C₂-C₂₅ alkenyl, C₃-C₂₅ cycloalkenyl, C₂-C₂₅ alkynyl, C₃-C₂₅ cycloalkynyl, C₁-C₂₅ alkoxyl, C₅-C₂₄ aryl, heteroaryl C₅-C₂₀, or a 3-12 membered heterocycle wherein the alkyl groups may be joined together in a ring, preferentially a hydrogen, a nitro group (—NO₂), a cyanide group (—CN), carboxyl (—COOH), ester (—COOR′), amido (—CONR′₂), sulphonyl (—SO₂R′), formyl (—CHO), sulphonoamido (—SO₂NR′₂), ketone (—COR′), in which R′ has the following meaning: C₁-C₅ alkyl, C₁-C₅ perfluoroalkyl, C₅-C₂₄ aryl;
R¹ denotes hydrogen, a fluoride atom, C₁-C₁₂ alkyl, C₃-C₁₂ cycloalkyl, C₂-C₁₂ alkenyl, C₃-C₁₂ cycloalkenyl, C₂-C₁₂ alkynyl, C₃-C₁₂ cycloalkynyl, C₁-C₁₂ alkoxyl, C₅-C₂₀ aryl, C₅-C₂₀ heteroaryl, or a 3-12 membered heterocycle;
with carbene complexes of ruthenium defined by Formulae 4a, 4b, 4c or 4d:

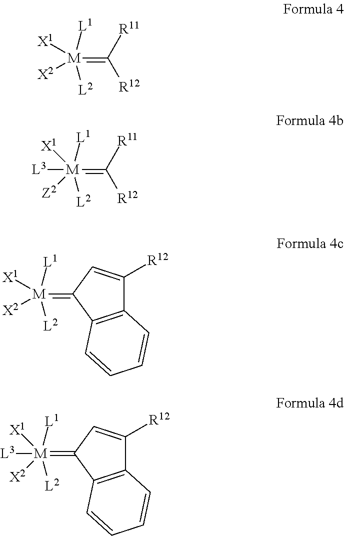

in which
M denotes ruthenium or osmium;
L¹, L² and L³, independently of one another, denote neutral ligands;
X¹ and X², independently of one another, denote an anionic ligand;
R¹¹ has the same meaning as R¹ of Formula 1;
R¹² denotes a hydrogen atom, C₅-C₂₀ aryl, C₅-C₂₀ heteroaryl, vinyl or allenyl.

Preferentially, the reaction is carried out over a period from 1 min. do 250 h, at a temperature in the range from 0 to 150° C.

Preferentially, the reaction is carried out in a chlorinated solvent or in aromatic hydrocarbons, or in protic or aprotic solvents, such as alcohols or ketones or in mixtures thereof.

Preferentially, the reaction is carried out in a solvent selected from among methylene chloride and/or toluene.

The present invention also relates to the use of complexes of ruthenium defined by Formula 1 as (pre)catalysts in metathesis reactions.

Preferentially, ruthenium complexes defined by Formula 1 are used as (pre)catalysts in metathesis ring closing reactions, homometathesis, cross-metathesis, “alkene-alkyne” metathesis (ene-yne), ROMP polymerisations as well as olefin cyclomerisation reactions.

The term “a fluoride atom” denotes an element selected from among F, Cl, Br, or I.

The term “carbene” denotes a molecule containing a neutral carbon atom with a valence number of two and two unpaired valence electrons. The term “carbene” also encompasses carbene analogues in which the carbon atom is substituted by another chemical elements such as boron, silicon, germanium, tin, lead, nitrogen, phosphorus, sulphur, selenium and tellurium.

The term “alkyl” refers to a saturated, linear, or branched hydrocarbon substituent with the indicated number of carbon atoms. Examples of an alkyl substituent are -methyl, -ethyl, -n-propyl, -n-butyl, -n-pentyl, -n-hexyl, -n-heptyl, -n-octyl, -n-nonyl, and -n-decyl. Representative branched —(C₁-C₁₀)alkyls encompass -isopropyl, -sec-butyl, -isobutyl, -tert-butyl, -isopentyl, -neopentyl, -1-methylbutyl, -2-methylbutyl, -3-methylbutyl, -1,1-dimethylpropyl, -1,2-dimethylpropyl, -1-methylpentyl, -2-methylpentyl, -3-methylpentyl, -4-methylpentyl, -1-ethylbutyl, -2-ethylbutyl, -3-ethylbutyl, -1,1-dimethylbutyl, -1,2-dimethylbutyl, -1,3-dimethylbutyl, -2,2-dimethylbutyl, -2,3-dimethylbutyl, -3,3-dimethylbutyl, -1-methylhexyl, -2-methylhexyl, -3-methylhexyl, -4-methylhexyl, -5-methylhexyl, -1,2-dimethylpentyl, -1,3-dimethylpentyl, -1,2-dimethylhexyl, -1,3-dimethylhexyl, -3,3-dimethylhexyl, -1,2-dimethylheptyl, -1,3-dimethylheptyl, and -3,3-dimethylheptyl and the like.

The term “alkoxyl” refers to an alkyl substituent as defined above attached via an oxygen atom.

The term “perfluoroalkyl” denotes an alkyl group as defined above in which all hydrogen atoms have been replaced by identical or different fluoride atoms.

The term “cycloalkyl” refers to a saturated mono- or polycyclic hydrocarbon substituent with the indicated number of carbon atoms. Examples of cycloalkyl substituents are -cyclopropyl, -cyclobutyl, -cyclopentyl, -cyclohexyl, -cycloheptyl, -cyclooctyl, -cyclononyl, -cyclodecyl, and the like.

The term “alkenyl” refers to an unsaturated, linear, or branched acyclic hydrocarbon substituent with the indicated number of carbon atoms and containing at least one double carbon-carbon bond. Examples of alkenyl substituents are: -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, -2-hexenyl, -3-hexenyl, -1-heptenyl, -2-heptenyl, -3-heptenyl, -1-octenyl, -2-octenyl, -3-octenyl, -1-nonenyl, -2-nonenyl, -3-nonenyl, -1-decenyl, -2-decenyl, -3-decenyl and the like.

The term “cycloalkenyl” refers to an unsaturated mono- or polycyclic hydrocarbon substituent with the indicated number of carbon atoms and containing at least one double carbon-carbon bond. Examples of cycloalkenyl substituents are -cyclopentenyl, -cyclopentadienyl, -cyclohexenyl, -cyclohexadienyl, -cycloheptenyl, -cycloheptadienyl, -cycloheptatrienyl, -cyclooctenyl, -cyclooctadienyl, -cyclooctatrienyl, -cyclooctatetraenyl, -cyclononenyl, -cyclononadienyl, -cyclodecenyl, -cyclodekadienyl and the like.

The term “alkynyl” refers to an unsaturated, linear, or branched acyclic hydrocarbon substituent with the indicated number of carbon atoms and containing at least one triple carbon-carbon bond. Examples of alkynyl substituents are -acethylenyl, -propynyl, -1-butynyl, -2-butynyl, -1-pentynyl, -2-pentynyl, -3-methyl-1-butynyl, -4-pentynyl, -1-hexynyl, -2-hexynyl, -5-hexynyl and the like.

The term “cycloalkynyl” refers to saturated mono- or polycyclic hydrocarbon substituent with the indicated number of carbon atoms and containing at least one triple carbon-carbon bond.

Examples of cycloalkynyl substituents are -cyclohexynyl, -cycloheptynyl, -cyclooctynyl, and the like.

The term “aryl” refers to an aromatic mono- or polycyclic hydrocarbon substituent with the indicated number of carbon atoms. Examples of aryl substituents are -phenyl, -tolyl, -xylyl, -naphthyl and the like.

The term “heteroaryl” refers to an aromatic mono- or polycyclic hydrocarbon substituent with the indicated number of carbon atoms in which at least one the carbon atom has been replaced by a heteroatom selected from among O, N and S. Examples of heteroaryl substituents are -furyl, -thienyl,-imidazolyl, -oxazolyl, -thiazolyl, -isoxazolyl, -triazolyl, -oxadiazolyl, -thiadiazolyl, -tetrazolyl, -pirydyl, -pirymidyl, -triazynyl, -indolyl, -benzo[b]furyl, -benzo[b]thienyl, -indazolyl, -benzoimidazolyl, -azaindolyl, -quinolyl, -isoquinolyl, -carbazolyl and the like.

The term “heterocycle” refers to saturated or partially unsaturated mono- or polycyclic hydrocarbon substituents, with the indicated number of carbon atoms in which at least one the carbon atom has been replaced by heteroatom selected from among O, N and S. Examples of heterocyclic substituents are -furyl, -thiophenyl, -pyrolyl, -oxazolyl, -imidazolyl, -thiazolyl, -isoxazolyl, -pirazolyl, -isothiazolyl, -triazynyl, -pyrolidynonyl, -pyrolidynyl, -hydantoinyl, -oxiranyl, -oxethanyl, -tetrahydrofuranyl, -tetrahydrothiophenyl, -quinolinyl, -isoquinolinyl, -chromonyl, -cumarynyl, -indolyl, -indolizynyl, -benzo[b]furanyl, -benzo[b]thiophenyl, -indazolyl, -purynyl, -4H-quinolizynyl, -isoquinolyl, -quinolyl, -phthalazynyl, -naphthyrydynyl, -carbazolyl, -β-carbolinyl and the like.

The term “neutral ligands” refers to uncharged substituents, capable of coordinating with a metallic centre (ruthenium or osmium atom). Examples of such ligands may be: amines, phosphines and their oxides, alkyl and alkane phosphorines and phosphoranes, arsines and their oxides, ethers, alkyl and aryl sulphides, coordinated hydrocarbons, alkyl and aryl halides.

The term “indenyl” refers to an unsaturated hydrocarbon substituent with an inden skeleton (benzocyclopentadiene).

The term “heteroindenyl” refers to an indenyl substituent, defined above in which at least one carbon atom is replaced with a heteroatom from a group encompassing: nitrogen, oxygen and sulphur.

The term “an anionic ligand” refers to a substituent capable of coordinating with a metallic centre (ruthenium atom) possessing a charge capable of the partial or full compensation of the metallic centre charge. Examples of such ligands may be: fluoride, chloride, bromide, iodide, cyanide, cyanate and thiocyanate anions, carboxylic acid anions, alcohol anions, anions of phenols, thiols and thiophenols, anions of hydrocarbons with a displaced charge (i.e. cyclopentadiene), anions of (organo)sulphuric and (organo)phosphoric acids as well as their esters (such as i.e. anions of alkylsulphonic and arylsulphonic acids, anions of alkylphosphoric and arylphosphoric acids, anions of alkyl and aryl esters of sulphuric acid, anions of alkyl and aryl esters of phosphoric acids, anions of alkyl and aryl esters of alkylphosphoric and arylphosphoric acids). Possibly, an anionic ligand may possess linked L¹, L², L³ groups such as a katechol anion, an acetylacetone anion, a salicylic aldehyde anion. Anionic ligands (X¹, X²) as well as neutral ligands (L¹, L², L³) may be linked forming polydentate ligands, for example: bidentate ligands (X¹, X²), tridentate ligands (X¹, X², L¹), tetradentate ligands (X¹, X², L¹, L²), bidentate ligands (X¹, L¹), tridentate ligands (X¹, L¹, L²), tetradentate ligands (X¹, L¹, L², L³), bidentate ligands (L¹, L²), tridentate ligands (L¹, L², L³). Examples of such ligands are: a katechol anion, an aceylacetone anion as well as a salicylic aldehyde anion.

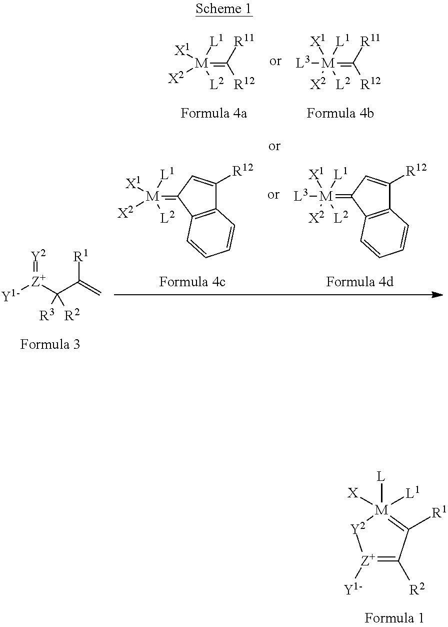

The examples below explain the production and use of the novel complexes.

### EXAMPLE I

### Synthesis of a catalyst defined by Formula 1a (according to Scheme I)

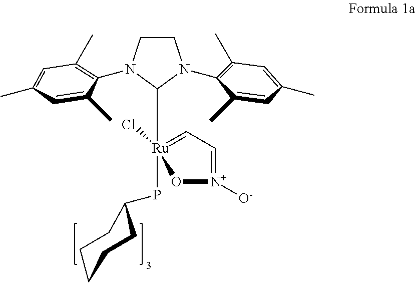

Using a protective argon atmosphere in a Schlenk vessel, we placed a solid carbene metal complex defined by Formula 4a, in which M denotes ruthenium, X¹ and X² denote chlorine, L¹ denotes tricyclohexylphosphine (PCy₃), L² denotes the NHC ligands defined by Formula 2a, in which R⁶ and R⁹ denote 2,4,6-trimethylphenyl, R⁷, R⁸ as well as R¹¹ are hydrogen and R¹² is phenyl (so-called Grubbs II-generation catalyst, 102 mg, 0.12 mmol), we added dry deoxygenated dichloromethane (2 ml). Next, we added the compound defined by Formula 3a:

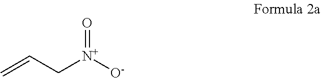

(13.1 mg, 0.15 mmol). The resulting solution were mixed at room temperature for 20 hours. From this time, all subsequent operations were performed in the open air, without the need for a protective argon atmosphere. The reaction mixture was concentrated in an evaporator and loaded onto a chromatography column packed with a silica gel. The column was developed with an ethyl acetate-cyclohexane solution (10% v/v), collecting the green fraction. After evaporating off the solvent, we obtained complex 1a as an olive, microcrystalline solid (52.6 mg, 55% yield).

¹H NMR (500 MHz, CDCl₃): δ=14.27 (d, J=3 Hz, 1H), 7.02-6.90 (m, 4H), 6.42 (d, J=3 Hz, 1H), 3.88-3.86 (m, 2H), 3.82-3.79 (m, 2H), 2.59 (s, 3H), 2.52 (s, 3H), 2.46 (s, 3H), 2.33 (s, 3H), 2.31 (s, 3H) 1.98 (s, 3H), 1.75-1.56 (m, 21H), 1.11-1.00 (m, 9H), 0.92-0.85 (m, 3H);

¹³C NMR (125 MHz, CDCl₃): δ=249.2, 219.3, 218.7, 138.8, 138.6, 138.4, 138.0, 137.6, 137.5, 136.3, 133.8, 130.4, 130.0, 129.9, 129.1, 128.9, 51.6, 51.2, 35.6, 35.1, 33.1, 33.0, 29.3, 28.9, 27.8, 27.7, 27.6, 27.5, 27.0, 26.5, 26.3, 26.1, 21.2, 21.1, 19.3, 18.7, 18.6, 16.9;

³¹P NMR (202 MHz, CDCl₃): δ=34.2 (s, 1P);

IR (KBr): ν=2925, 2850, 1813, 1512, 1483, 1430, 1379, 1266, 1169, 1041, 849, 743 cm⁻¹

MS (FD/FI): m/z found for the formula C₄₁H₆₁³⁵ClN₃O₂P¹⁰²Ru: 795.3 (M+).

### EXAMPLE II

### Synthesis of a Catalyst Defined by Formula 1b (According to Scheme I)

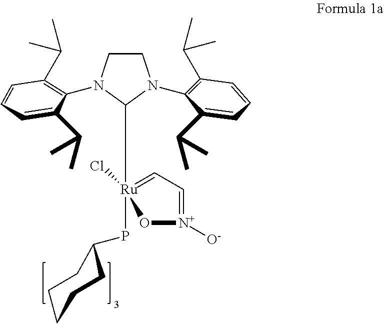

Using a protective argon atmosphere in a Schlenk vessel a solid carbene metal complex defined by Formula 4a, in which M denotes ruthenium, X¹ and X² denote chlorine, L¹ denotes tricyclohexylphosphine (PCy₃), L² denotes the NHC ligands defined by Formula 2a, in which R⁶ and R⁹ denote 2,6-di(2-propyl)phenyl, R⁷, R⁸ as well as R¹¹ are hydrogen and R¹² phenyl (149 mg, 0.16 mmol), we added dry deoxygenated dichloromethane (2 ml). Next, we added the compound defined by Formula 3a (17.4 mg, 0.20 mmol). The resulting solution were mixed at room temperature for about 15 min. From this time, all subsequent operations were performed in the open air, without the need for a protective argon atmosphere. The reaction mixture was concentrated in an evaporator and loaded onto a chromatography column packed with a silica gel.

The column was developed with an ethyl acetate-cyclohexane solution (10% v/v), collecting the green fraction. After evaporating off the solvent, we obtained complex 1b as an olive, microcrystalline solid (93.3 mg, 66% yield).

¹H NMR (600 MHz, CDCl₃): δ=13.81 (d, J=3 Hz, 1H), 7.36-7.10 (m, 6H), 6.29 (d, J=3 Hz, 1H), 4.20-4.10 (m, 1H), 4.10-4.00 (m, 1H), 4.00-3.80 (m, 3H), 3.75-3.65 (m, 1H), 3.65-3.55 (m, 1H), 2.70-2.64 (m, 1H), 1.70-1.64 (m, 3H), 1.60-1.50 (m, 18H), 1.39-1.35 (m, 3H), 1.26-1.19 (m, 10H), 1.15-1.08 (m, 9H), 1,07-0.92 (m, 14H);

¹³C NMR (150 MHz, CDCl₃): δ=246.4, 222.2, 221.7, 148.64, 148.60, 148.5, 147.4, 137.5, 135.1, 130.0, 129.7, 129.0, 125.2, 124.2, 124.1, 123.9, 77.2, 77.0, 76.8, 54.0, 53.7, 33.2, 33.0, 29.6, 28.7, 28.5, 28.3, 27.9, 27.8, 27.2, 27.2, 26.9, 26.6, 26.3, 26.1, 23.4, 22.8, 22.0;

³¹P NMR (202 MHz, CDCl₃): δ=35.3 (s, 1P);

IR (KBr): ν=2962, 2927, 2851, 1431, 1414, 1383, 1326, 1269, 1238, 1170, 1047, 803, 758, 734 cm⁻¹;

MS (FD/FI): m/z found for the formula C₄₇H₇₃³⁵ClN₃O₂P¹⁰²Ru: 879.3 (M⁺).

X-ray structural analysis for compound 1b:

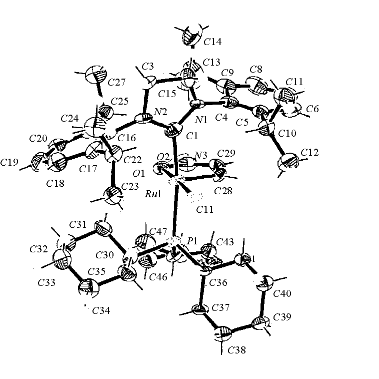

### EXAMPLE III

### Synthesis of a Catalyst Defined by Formula 1c (According to Scheme I)

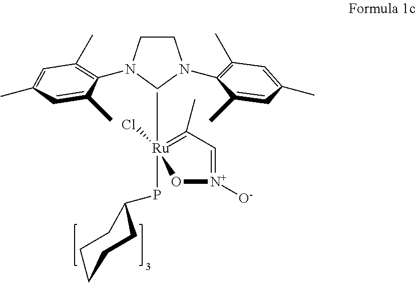

Using a protective argon atmosphere in a Schlenk vessel a solid carbene metal complex defined by Formula 4a, in which M denotes ruthenium, X¹ and X² denote chlorine, L¹ denotes tricyclohexylphosphine (PCy₃), L² denotes the NHC ligands defined by Formula 2a, in which R⁶ and R⁹ denote 2,4,6-trimethylphenyl, R⁷, R⁸ as well as R¹¹ are hydrogen and R¹² is phenyl (so-called Grubbs II-generation catalyst, 20.7 mg, 0.024 mmol), we added dry deoxygenated dichloromethane (0.3 ml). Next, we added the compound defined by Formula 3b:

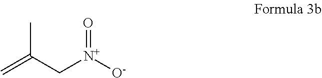

(5 mg, 0.049 mmol). The resulting solution were mixed at room temperature for 20 hours. From this time, all subsequent operations were performed in the open air, without the need for a protective argon atmosphere. The reaction mixture was concentrated in an evaporator and loaded onto a chromatography column packed with a silica gel. The column was developed with an ethyl acetate-cyclohexane solution (10% v/v), collecting the green fraction. After evaporating off the solvent, we obtained complex 1c as an olive, microcrystalline solid (9.5 mg, 50% yield).

¹H NMR (500 MHz, CDCl₃): δ=7.03 (s, 1H), 6.93 (s, 1H), 6.92 (s, 1H), 6.88 (s, 1H), 6.65 (s, 1H), 4.06-3.97 (m, 1H), 3.88-3.72 (m, 3H), 2.59 (s, 3H), 2.54 (s, 3H), 2.46 (s, 3H), 2.31 (s, 6H), 2.00 (s, 3H), 1.91 (s, 3H), 1.75-1.54 (m, 16H), 1.30-1.00 (m, 15H), 0.92-0.81 (m, 3H);

¹³C NMR (125 MHz, CDCl₃): δ=271.1, 271.0, 217.9, 217.2, 139.0, 138.7, 138.6, 138.3, 138.2, 138.0, 136.6, 133.6, 129.91, 129.85, 129.4, 128.6, 51.9, 51.5, 35.2, 33.6, 33.4, 28.9, 28.8, 27.9, 27.8, 27.6, 27.5, 26.9, 26.5, 21.1, 21.0, 19.2, 18.7, 18.5, 16.6;

³¹P NMR (202 MHz, CDCl3): δ=27.0 (s, 1P);

IR (film z CHCl₃): ν=2927, 2851, 1481, 1444, 1268, 1185, 850, 752, 624 cm⁻¹;

MS (FD/FI): m/z found for the formula C₄₂H₆₃³⁵ClN₃O₂P¹⁰²Ru:809.2 (M⁺).

### EXAMPLE IV

### Synthesis of a Catalyst Defined by Formula Id (According to Scheme I)

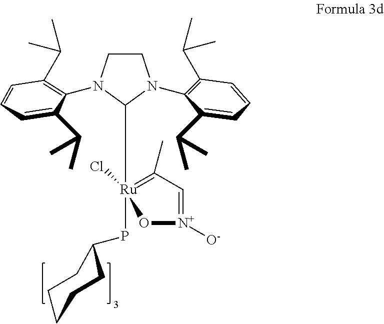

Using a protective argon atmosphere in a Schlenk vessel a solid carbene metal complex defined by Formula 4a, in which M denotes ruthenium, X¹ and X² denote chlorine, L¹ denotes tricyclohexylphosphine (PCy₃), L² denotes the NHC ligands defined by Formula 2a, in which R⁶ and R⁹ denote 2,6-di(2-propyl)phenyl, R⁷, R⁸ as well as R¹¹ are hydrogen and R¹² phenyl (168 mg, 0.18 mmol), we added dry deoxygenated dichloromethane (2 ml). Next we added the compound defined by Formula 3b (22.7 mg, 0.23 mmol). The resulting solution were mixed at room temperature for about 15 min. From this time, all subsequent operations were performed in the open air, without the need for a protective argon atmosphere. The reaction mixture was concentrated in an evaporator and loaded onto a chromatography column packed with a silica gel. The column was developed with an ethyl acetate-cyclohexane solution (10% v/v), collecting the green fraction. After evaporating off the solvent, we obtained complex 1d as an olive, microcrystalline solid (83.1 mg, 52% yield).

¹H NMR (600 MHz, CDCl₃): δ=7.40-7.10 (m, 6H), 6.67 (s, 1H), 4.10-4.00 (m, 1H), 3.98-3.87 (m, 2H), 3.68-3.54 (m, 3H), 3.75-3.65 (m, 1H), 3.65-3.55 (m, 1H), 2.41-2.32 (m, 1H), 2.16 (s, 3H), 1.77 (s, 3H), 1.69-1.59 (m), 1.57-1.48 (m), 1.39-1.29 (m), 1.25-1.20 (m), 1.20-1.12 (m), 1.11-1.02 (m), 1.01-0.91 (m).

¹³C NMR (150 MHz, CDCl₃): 6=268.3, 219.5, 219.0, 149.3, 148.8, 148.5, 147.3, 138.1, 130.4, 129.9, 128.9, 124.8, 124.2, 123.2, 77.2, 77.0, 76.8, 55.0, 53.9, 55.4, 35.3, 33.4, 30.9, 29.2, 28.8, 28.6, 28.2, 27.95, 27.88, 27.8, 27.1, 26.95, 26.87, 26.6, 26.4, 26.1, 25.9, 23.9, 23.2, 22.3, 21.7; ³¹P NMR (202 MHz, CDCl₃): δ=26.4 (s, 1P);

IR (film z CHCl₃): ν=2962, 2928, 2851, 1436, 1414, 1268, 1234, 1185, 803, 756, 616 cm⁻¹

MS (FD/FI): m/z found for the formula C₄₉H₇₅³⁵ClN₃O₂P¹⁰²Ru: 893.4 (M⁺).

Examples of uses of compound 1 as catalyst in the metathesis reactions with ring closure, cross-metathesis, “alkene-alkyne” metathesis (ene-yne), as well as the olefin cycloisomerisation reaction.

### EXAMPLE V

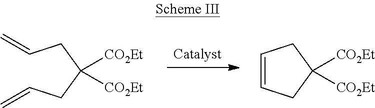 [SMILES: C=CCC(CC=C)(C(=O)OCC)C(=O)OCC ; CCOC(=O)C1(C(=O)OCC)CC=CC1]

Procedure A: in a Schlenk vessel, we placed a diene solution (48.4 mg, 0.20 mmol) in toluene (2 ml), we added hexachloroethane (1.9 mg, 4%ₘₒₗ), and next, the catalyst 1a (1.6 mg, 1%ₘₒₗ). The vessel contents were mixed at a temperature of 80° C. for 2 h. The raw post-reaction mixture was analysed using gas chromatography. The yield of the metathesis reactions was 100%.

Procedure B: in a Schlenk vessel, we placed a diene solution (48.0 mg, 0.20 mmol) in toluene (2 ml), we added chlorotrimethylsilane (0.9 mg, 4%ₘₒₗ), and next, the catalyst 1a (1.6 mg, 1%ₘₒₗ). The vessel contents were mixed at a temperature of 80° C. for 2 h. The raw post-reaction mixture was analysed using gas chromatography. The efficiency of the product metathesis was 85%.

Procedure C: in a Schlenk vessel, we placed a diene solution (31.2 mg, 0.13 mmol) in carbon tetrachloride (0.6 ml), and next we added catalyst 1b (5.1 mg, 5%ₘₒₗ). The vessel contents were mixed at a temperature of 60° C. for 4 h. The raw post-reaction mixture was analysed using gas chromatography. The yield of the metathesis reactions was 98%.

Procedure D: in a Schlenk vessel, we placed a diene solution (30.7 mg, 0.13 mmol) in carbon tetrachloride (0.6 ml), and next we added catalyst 1a (5.0 mg, 5%ₘₒₗ). The vessel contents were mixed at a temperature of 60° C. for 2 h. The raw post-reaction mixture was analysed using gas chromatography. The yield of the metathesis reactions was 100%.

### EXAMPLE VI

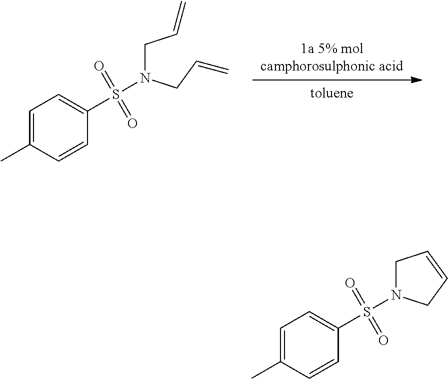 [SMILES: C=CCN(CC=C)S(=O)(=O)c1ccc(C)cc1 ; Cc1ccc(S(=O)(=O)N2CC=CC2)cc1]

In a Schlenk vessel, we placed a diene solution (74.1 mg, 0.29 mmol) in toluene (1.5 ml), we added camphorosulphonic acid (3.7 mg, 5%ₘₒₗ), and next, the catalyst 1a (12 mg, 5%ₘₒₗ). The vessel contents were mixed at a temperature of 80° C. for 29 h. The raw post-reaction mixture was analysed using gas chromatography. The conversion of the metathesis reactions was 99%.

### EXAMPLE VII

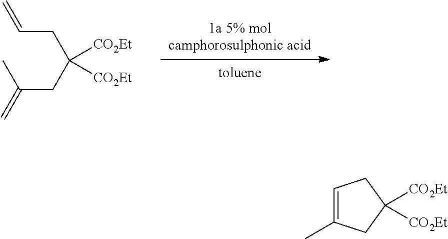 [SMILES: C=CCC(CC(=C)C)(C(=O)OCC)C(=O)OCC ; CCOC(=O)C1(C(=O)OCC)CC=C(C)C1]

In a Schlenk vessel, we placed a diene solution (77.9 mg, 0.31 mmol) in toluene (1.5 ml), we added camphorosulphonic acid (5.1 mg, 7%ₘₒₗ), and next, the catalyst 1a (11.9 mg, 5%ₘₒₗ). The vessel contents were mixed at a temperature of 80° C. for 14 h. The raw post-reaction mixture was analysed using gas chromatography. The conversion of the metathesis reactions was 100%.

### EXAMPLE VIII

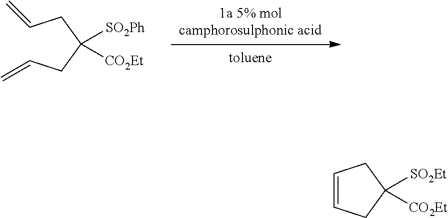

In a Schlenk vessel, we placed a diene solution (92.7 mg, 0.31 mmol) in toluene (1.5 ml), we added camphorosulphonic acid (4.4 mg, 6%ₘₒₗ), and next, the catalyst 1a (12.0 mg, 5%ₘₒₗ). The vessel contents were mixed at a temperature of 80° C. for 2 h. The raw post-reaction mixture was analysed using gas chromatography. The conversion of the metathesis reactions was 100%.

### EXAMPLE IX

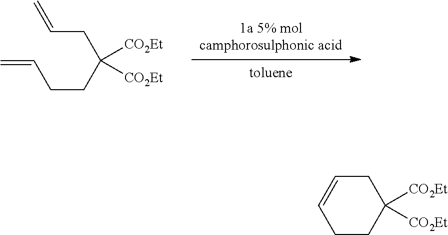 [SMILES: C=CCCC(CC=C)(C(=O)OCC)C(=O)OCC ; CCOC(=O)C1(C(=O)OCC)CC=CCC1]

In a Schlenk vessel, we placed a diene solution (76.0 mg, 0.31 mmol) in toluene (1.5 ml), we ,C₃ added camphorosulphonic acid (4.6 mg, 7%ₘₒₗ), and next, the catalyst 1a (11.9 mg, 5%ₘₒₗ). The vessel contents were mixed at a temperature of 80° C. for 3 h. The raw post-reaction mixture was analysed using gas chromatography. The conversion of the metathesis reactions was 100%.

### EXAMPLE X

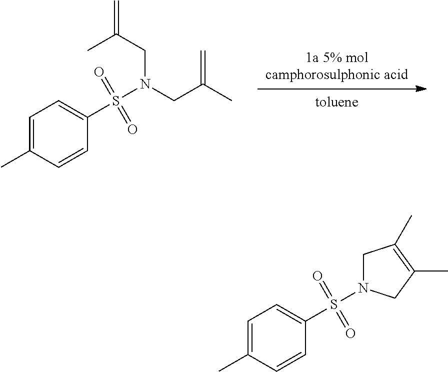

In a Schlenk vessel, we placed a diene solution (83.4 mg, 0.30 mmol) in toluene (1.5 ml), we added camphorosulphonic acid (3.8 mg, 5%ₘₒₗ), and next, the catalyst 1a (11.9 mg, 5%ₘₒₗ). The vessel contents were mixed at a temperature of 80° C. for 53 h. The raw post-reaction mixture was analysed using gas chromatography. The conversion of the metathesis reactions was 84%.

### EXAMPLE XI

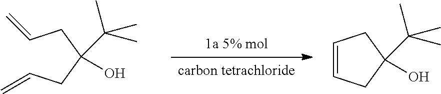

In a Schlenk vessel, we placed a diene solution (50.3 mg, 0.30 mmol) in carbon tetrachloride (1.5 ml), and next we added catalyst 1a (12.2 mg, 5%ₘₒₗ). The vessel contents were mixed at a temperature of 65° C. for 3 h. The raw post-reaction mixture was analysed using gas chromatography. The conversion of the metathesis reactions was 100%.

### EXAMPLE XII

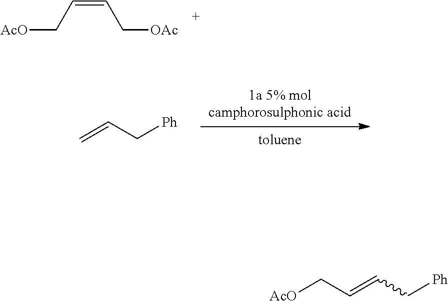

In a Schlenk vessel, we placed 1,4 diacetoxybut-2ene (110.0 mg, 0.64 mmol) and allylbenzene (35.8 mg, 0.30 mmol) in toluene (1.5 ml), we added camphorosulphonic acid (4.7 mg, 7%ₘₒₗ), and next, the catalyst 1a (12.1 mg, 5%ₘₒₗ). The vessel contents were mixed at a temperature of 80° C. for 29 h. The raw post-reaction mixture was analysed using gas chromatography. The yield of the cross-metathesis reaction was 41%.

### EXAMPLE XIII

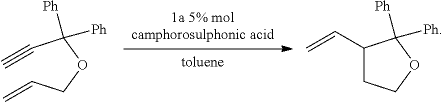 [SMILES: C#CC(OCC=C)(c1ccccc1)c1ccccc1 ; C=CC1CCOC1(c1ccccc1)c1ccccc1]

In a Schlenk vessel, we placed an enyne solution (76.1 mg, 0.31 mmol) in toluene (1.5 ml), we added camphorosulphonic acid (4.5 mg, 6%ₘₒₗ), and next, the catalyst 1a (12.4 mg, 5%ₘₒₗ). The vessel contents were mixed at a temperature of 80° C. for 24 h. The raw post-reaction mixture was analysed using gas chromatography. The conversion of the metathesis reactions was 100%.

### EXAMPLE XIV

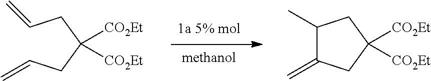 [SMILES: C=CCC(CC=C)(C(=O)OCC)C(=O)OCC ; C=C1CC(C(=O)OCC)(C(=O)OCC)CC1C]

In a Schlenk vessel, we placed a diene solution (73.5 mg, 0.31 mmol) in methanol (1.5 ml), and next we added catalyst 1a (11.7 mg, 5%ₘₒₗ). The vessel contents were mixed at a temperature of 65° C. for 42 h. The raw post-reaction mixture was analysed using gas chromatography. The yield of the cycloisomerisation reaction was 82%.

### EXAMPLE XV

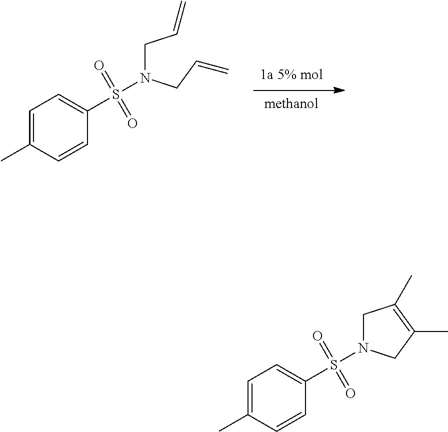

In a Schlenk vessel, we placed a diene solution (77.4 mg, 0.31 mmol) in methanol (1.5 ml), and next we added catalyst 1a (11.9 mg, 5%ₘₒₗ). The vessel contents were mixed at a temperature of 65° C. for 50 h. The raw post-reaction mixture was analysed using gas chromatography. The yield of the cycloisomerisation reaction was 88%.

### EXAMPLE XVI

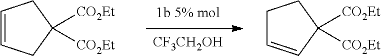 [SMILES: CCOC(=O)C1(C(=O)OCC)CC=CC1 ; "CF3CH2OH" ; CCOC(=O)C1(C(=O)OCC)C=CCC1]

In a Schlenk vessel, we placed an olefin solution (54.1 mg, 0.25 mmol) in trifluoroethanol (2 ml), and next we added catalyst 1a (10.8 mg, 5%ₘₒₗ). The vessel contents were mixed at a temperature of 65° C. for 71 h. The raw post-reaction mixture was analysed using gas chromatography. The yield of the isomerisation reaction was 77%.

### EXAMPLE XVII

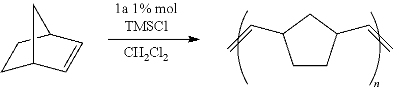

n

In a Schlenk vessel, we placed a norbornene solution (187 mg, 1.4 mmol) in dichloromethane (5 ml) and were mixed at a temperature of 40° C. Next, we added chlorotrimethylsilane (6.1 mg, 4%ₘₒₗ) and catalyst 1a (11.1 mg, 1%ₘₒₗol). The vessel contents were mixed at the same temperature for 10 min, whereafter this was poured into another vessel containing 15 ml of methanol and a white solid was precipitated which was filtered out and dried under reduced pressure over a vacuum pump. We obtained a product (119 mg, 90% yield) in the form of a white solid.

### EXAMPLE XVIII

Production of polidicyclopentadiene: a flask was loaded with dicyclopentadiene (132 mg, 1.0 mmol) in toluene (5 mL) and mixed at room temperature. Next, we added a chlorotrimethylsilane solution (1.1 mg, 1%ₘₒₗ) and catalyst 1a (0.2 mg, 0.025%ₘₒₗ in toluene and the flask contents were mixed at the same temperature for 10 min. Next, we supplemented the flask with toluene and brought it to boiling temp. in order to wash off the unreacted dicyclopentadiene. The insoluble polymer was washed with toluene and dried under reduced pressure at a temperature of 100° C. for 12 h. The conversion of dicyclopentadiene was 99%.

### EXAMPLE XIX

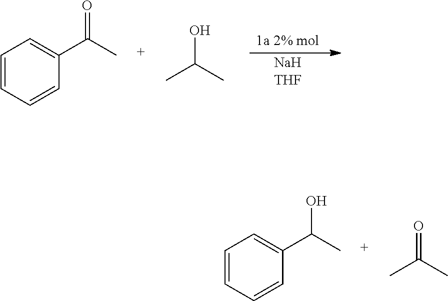

In a Schlenk vessel, we placed a solution of catalyst 1a (15.7 mg, 2%ₘₒₗ) in tetrahydrofuran (2.5 ml) and we added sodium hydride (2.8 mg, 7%ₘₒₗ). To this mixture we then added acetophenone (120.3 mg, 1.0 mmol) and isopropyl alcohol (2.5 ml). The vessel contents were mixed at a temperature of 70° C. for 5 h. The raw mixture was purified using column chromatography on a silica gel (elution with cyclohexane: ethyl acetate 20:1). We obtained 95 mg of a liquid product (yield 78%).

1. A metal complex defined by Formula 1: 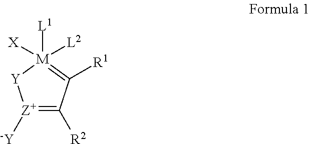
in which:
M denotes ruthenium or osmium;
L¹ and L² denote neutral ligands;
X denotes an anionic ligand;
Z denotes a nitrogen atom;
Y denotes an oxygen atom;
R¹, R² denote, independently of one another, a hydrogen atom, a fluoride atom, C₁-C₂₅ alkyl, C₁-C₂₅ perfluoroalkyl, C₂-C₂₅ alkene, C₃-C₇ cycloalkyl, C₂-C₂₅ alkenyl, C₃-C₂₅ cycloalkenyl, C₂-C₂₅ alkynyl, C₃-C₂₅ cycloalkynyl, C₁-C₂₅ alkoxyl, C₅-C₂₄ aryl, heteroaryl C₅-C₂₀, or a 3-12 membered heterocycle wherein the alkyl groups may be joined together in a ring, preferentially a hydrogen, a nitro group (—NO₂), a cyanide group (—CN), carboxyl (—COOH), carboxyl (—COOR′), amido (—CONR′₂), sulphonyl (—SO₂R′), formyl (—CHO), sulphonoamido (—SO₂NR′₂), ketone (—COR′), in which R′ has the following meaning: C₁-C₅ alkyl, C₁-C₅ perfluoroalkyl, C₅-C₂₄ aryl.

2. The complex according to claim 1, characterised in that the anionic ligand X denotes a fluoride atom, a —CN, —SCN, —OR⁴, —SR⁴, —O(C═O)R⁴, —O(SO₂)R⁴, —OSiR₃⁴, where R⁴ denotes C₁-C₁₂ alkyl, C₃-C₁₂ cycloalkyl, C₂-C₁₂ alkenyl, or C₅-C₂₀ aryl group, which may be substituted with at least one of C₁-C₁₂ alkyl, C₁-C₁₂ perfluoroalkyl, C₁-C₁₂ alkoxyl or a fluoride atom;
R¹ denotes a hydrogen atom or methyl group;
R² denotes a hydrogen atom;
neutral ligands L¹ and L² are selected, independently of one another, from a group encompassing —P(R⁵)₃, —P(OR⁵)₃ or N-heterocyclic carbene ligands denoted by Formulae 2a, 2b, 2c, 2d, 2e, 2f, 2g, 2h, 2i, 2j, 2k, 2l, 2m, 2n, 2o or 2p 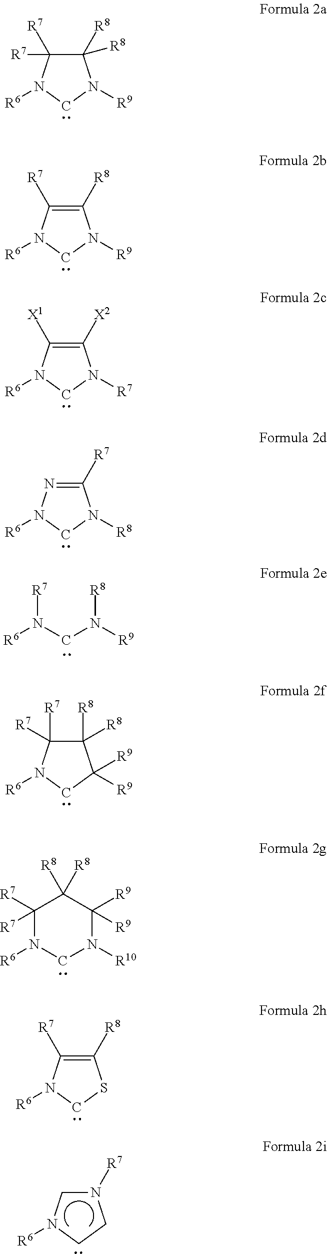 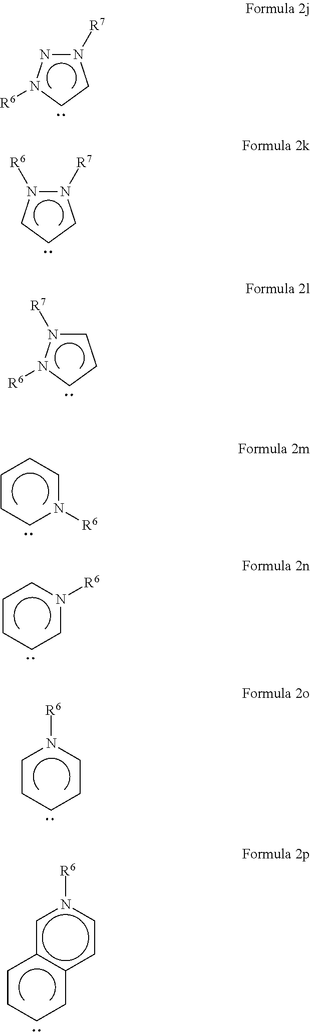
where:
each R⁵ denotes, independently of one another, C₁-C₁₂ alkyl, C₃-C₁₂ cycloalkyl, C₅-C₂₀ aryl, 5-12 membered heteroaryl;
each R⁶, R⁷, R⁸, R⁹ and R¹⁰ denotes, independently of one another, a hydrogen atom, C₁-C₁₂ alkyl, C₃-C₁₂ cycloalkyl, C₂-C₁₂ alkenyl or C₅-C₂₀ aryl which may be substituted with at least one C₁-C₁₂ alkyl, C₁-C₁₂ perfluoroalkyl, C₁-C₁₂ alkoxyl or fluoride atom, and groups R⁶, R⁷, R⁸, R⁹ and R¹⁰ may possibly be interconnected.

3. The complex according to claim 1, characterised in that X denotes a chlorine atom;
R¹ denotes a hydrogen atom or methyl group;
R² denotes a hydrogen atom
neutral ligand L¹ denotes —P(R⁵)₃ in which substituent R⁵ has the same meaning as defined above; and
neutral ligand L² denotes ligands defined by Formula 2a or 2b: 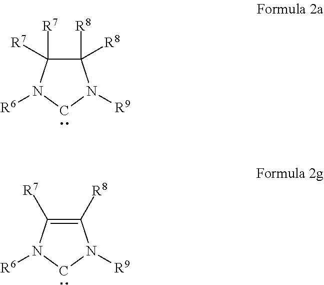
in which substituents R⁶, R⁷, R⁸ and R⁹ mean as defined above.

4. A method of producing a the ruthenium complex defined in claim 1, characterised in that the compound defined by Formula 3 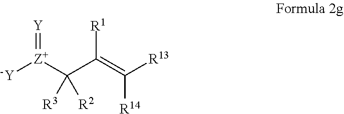
in which R¹, R², Z, Y¹, Y² have meanings as defined above, whereas R³, R¹³, R¹⁴ denote, independently of one another, a hydrogen atom, a fluoride atom, a C₁-C₂₅ alkyl, C₁-C₂₅ perfluoroalkyl, C₂-C₂₅ alkene, C₃-C₇ cycloalkyl, C₂-C₂₅ alkenyl, C₃-C₂₅ cycloalkenyl, C₂-C₂₅ alkynyl, C₃-C₂₅ cycloalkynyl, C₁-C₂₅ alkoxyl, C₅-C₂₄ aryl, heteroaryl C₅-C₂₀, or a 3-12 membered heterocycle wherein the alkyl groups may be joined together in a ring, preferentially a hydrogen, a nitro group (—NO₂), a cyanide group (—CN), a carboxyl (—COOH), ester (—COOR′), amido (—CONR′₂), sulphonyl (—SO₂R′), formyl (—CHO), sulphonoamido (—SO₂NR′₂), or ketone (—COR′) group, in which R has the following meaning: C₁-C₅ alkyl, C₁-C₅ perfluoroalkyl or C₅-C₂₄ aryl;
R¹ denotes a hydrogen, a fluoride atom, a C₁-C₁₂ alkyl, C₃-C₁₂ cycloalkyl, C₂-C₁₂ alkenyl, C₃-C₁₂ cycloalkenyl, C₂-C₁₂ alkynyl, C₃-C₁₂ cycloalkynyl, C₁-C₁₂ alkoxyl, C₅-C₂₀ aryl, C₅-C₂₀ heteroaryl, or a 3-12 membered heterocycle;
is reacted with carbene complexes of ruthenium defined by Formula 4a, 4b, 4c or 4d: 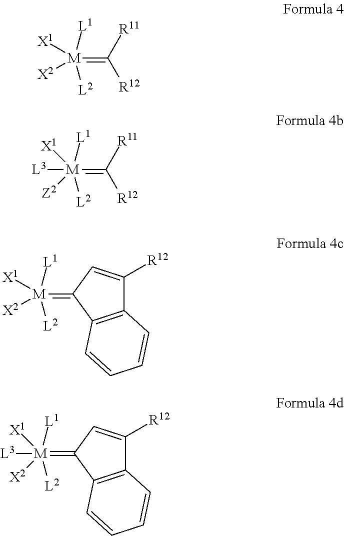
in which
M denotes ruthenium or osmium;
L¹, L² and L³, independently of one another, denote neutral ligands;
X¹ and X², independently of one another, denote an anionic ligand;
R¹¹ has the same meaning as R¹ of Formula 1;
R¹² denotes a hydrogen atom, C₅-C₂₀ aryl, C₅-C₂₀ heteroaryl, vinyl or allenyl.

5. The method according to claim 4, characterised in that the reaction is carried out over a period from 1 min. to 250 h, at a temperature of from 0 to 150° C.

6. The method according to claim 4, characterised in that the reaction is carried out in a protic or aprotic solvent, a chlorinated solvent or in an aromatic hydrocarbon solvent, or in mixtures thereof.

7. The method according to claim 4, characterised in that the reaction is carried out in a solvent selected from among methylene chloride and/or toluene.

8. A use the ruthenium complex defined by Formula 1 defined in claim 1, as a (pre)catalyst in metathesis processes, isomerisation and cycloisomerisation of olefins as well as in of the hydrogen transfer reaction.

9. The use according to claim 8, characterised in that complexes of ruthenium are used as (pre)catalysts in ring closing metathesis reactions, homometathesis, cross metathesis, “alkene-alkyne” metathesis (ene-yne) or in ROMP polymerisation reactions.

10. The use according to claim 9, characterised in that complexes of ruthenium are used as (pre)catalysts in a metathetic polymerisation with dicyclopentadiene ring opening.

11. The use according to claim 8, characterised in that the reaction is carried out in the presence of an acid or halide derivatives of alkanes and silanes or N-haloimides and amides.
